(19) United States
(12) Patent Application Publication
(10) Pub. No.: US 2025/0152968 A1
(43) Pub. Date: May 15, 2025

(54) THERAPEUTIC DEVICE

(71) Applicant: Shenzhen Guangshu Medical Technology Co., Ltd, Shenzhen (CN)

(72) Inventors: ALAIN DIJKSTRA, Amstelveen (NL); MICHAEL KASBERGEN, Schoonhoven (NL)

(73) Assignee: Shenzhen Guangshu Medical Technology Co., Ltd, Shenzhen (CN)

(21) Appl. No.: 18/533,235

(22) Filed: Dec. 8, 2023

(30) Foreign Application Priority Data
Nov. 10, 2023 (CN) 2023230451137

(51) Int. Cl. A61N 5/067; A61N 5/06

(52) U.S. Cl. CPC A61N 5/067; A61N 2005/0647; A61N 2005/0653; A61N 2005/0659; A61N 2005/0661; A61N 2005/0663

(57) ABSTRACT

Embodiments of the present invention provide a therapeutic device. The therapeutic device includes a first substructure including a Printed Circuit Board (PCB), a transmitter induction coil electrically coupled to the PCB, and a power source electrically coupled to the transmitter induction coil. The therapeutic device further includes one or more second substructures including one or more of respective stimulation elements and one or more respective receiver induction coils electrically coupled to the one or more respective stimulation elements. The one or more stimulation elements are configured to be facing towards a body portion of a user, when in use. Also, the one or more receiver induction coils are configured to receive power from a magnetic field generated by the transmitter induction coil.

### TECHNICAL FIELD

The present invention relates generally to therapeutic and recreational devices. More specifically, the present invention relates to devices that can deliver multiple kinds of stimulation such as electrical, heating, cooling, and irradiation while operating in multiple modes of operation.

### BACKGROUND ART

Stimulation devices of several kinds for therapeutic and recreational purposes have been known in the art for quite some time. Such devices use electrodes, heating elements, cooling elements, and irradiation sources such as Light Emitting Diodes (LEDs) and lasers to deliver energy in one form or the other to a portion of the body for pain relief, muscle relaxation, skin rejuvenation, neural stimulation etc. The stimulation devices that are currently known in the art are generally single-purpose, in a manner that they can only provide a single kind of stimulation. Although, in some solutions, a combination of two kinds of stimulation, such as irradiation and electrical stimulation, has been proposed, however, such solutions have very little scope for customization.

Moreover, currently available solutions are designed to operate in a very small number of pre-defined operating modes leaving little room for a user to adjust or modify operational characteristics of stimulation elements used. In addition, the currently available stimulation devices are power intensive and therefore require large batteries or circuits capable of converting Alternating Current (AC) to Direct Current, making the devices bulky in construction and difficult to handle. In cases where such devices are battery-powered, bulk to such devices is added by the wiring, connections and terminals that are required to electrically couple several stimulation elements with the batteries and other components of control circuitry, such as processors, memory units, and system buses.

Therefore, there is a need for a therapeutic device that overcomes the disadvantages and limitations associated with the prior art and provides a more satisfactory solution.

### OBJECTS OF THE INVENTION

Some of the objects of the invention are as follows:

An object of the present invention is to provide a therapeutic device that can be used for multiple purposes such as electrical stimulation, heating, cooling, vibratory massage, etc.

Another object of the present invention is to provide a therapeutic device that can operate in multiple predefined and user-customized modes of operation.

Another object of the present invention is to provide a therapeutic device that is power efficient and generates relatively minimal residual heat.

It is also an object of the present invention to provide a therapeutic device that is lighter in weight, simpler in construction, and capable of being carried and transported with relative ease.

### SUMMARY OF THE INVENTION

According to a first aspect of the present invention, there is provided a therapeutic device. The therapeutic device includes a first substructure including a Printed Circuit Board (PCB), a transmitter induction coil electrically coupled to the PCB, and a power source electrically coupled to the transmitter induction coil. The therapeutic device further includes one or more second substructures including one or more of respective stimulation elements and one or more respective receiver induction coils electrically coupled to the one or more respective stimulation elements. The one or more stimulation elements are configured to be facing towards a body portion of a user, when in use. Also, the one or more receiver induction coils are configured to receive power from a magnetic field generated by the transmitter induction coil.

In one embodiment of the invention, the transmitter induction coil defines a transmitter outer periphery. The one or more second substructures are located within the transmitter outer periphery, in one or more respective second planes parallel and/or coincident with a first plane of the transmitter outer periphery.

In one embodiment of the invention, the one or more stimulation elements is selected from a group consisting of irradiation sources, heating elements, cooling elements, vibration elements, electrodes and combinations thereof.

In one embodiment of the invention, the irradiation sources are selected from a group consisting of Light Emitting Diodes (LEDs), and lasers.

In one embodiment of the invention, the therapeutic device further includes a wearable element, such that, the one or more second substructures are installed with the wearable element.

In one embodiment of the invention, the first substructure is provided within a detachable housing configured to be detachably attached with the wearable element including the one or more second substructures.

In one embodiment of the invention, the wearable elements is embodied as a face mask.

In one embodiment of the invention, the wearable element is embodied as a wearable pad. The wearable pad includes an outer layer including a first surface facing towards the ambient and a second surface facing opposite to the first surface. Also, the wearable pad includes an inner layer coupled to the outer layer, the inner layer including a third surface facing towards the outer layer and a fourth surface configured to be facing towards the body portion of the user when in use. Furthermore, the one or more second substructures are located between the outer layer and the inner layer.

In several embodiments of the invention, the wearable pad is embodied as one or more of a headband, a neck belt, a body pad, a bandage, a waist belt, a shin guard, an ankle pad, a sole of a footwear, and combinations thereof.

In one embodiment of the invention, the inner layer includes one or more slots cut into the inner layer. The one or more second substructures are removably located in the one or more slots, such that, the one or more second substructures are configured to translate along the one or more slots.

In one embodiment of the invention, the one or more second substructures are configured to be translated along the one or more slots through manual manipulation, electrical actuation, and combinations thereof.

In one embodiment of the invention, the one or more slots are distributed in accordance with a predetermined distribution pattern comprising one or more sectors configured to overlap one or more respective body regions of the body portion.

In one embodiment of the invention, the inner layer is made up of a transparent silicone material.

In one embodiment of the invention, the first substructure further includes a mode selector electrically coupled to the PCB. The mode selector is configured to switch between a plurality of predefined operating modes of the one or more stimulation elements.

In one embodiment of the invention, the first substructure further includes a control module. The control module includes a processor and a memory unit operably connected to the processor. The memory unit includes machine-readable instructions that when executed by the processor, enable the processor to switch between a plurality of predefined operating modes of the one or more stimulation elements.

In one embodiment of the invention, for operating the one or more stimulation elements in an operating mode, the processor is further enabled to receive a control input corresponding to the operating mode, from a user interface, the control input configured to switch between a plurality of predefined operating modes of the one or more stimulation elements.

In one embodiment of the invention, for operating the one or more stimulation elements in an operating mode, the processor is further enabled to receive a control input corresponding to the operating mode, from a user computing device, via a communication interface of the control module, the control input configured to switch between a plurality of predefined operating modes of the one or more stimulation elements.

In one embodiment of the invention, the power source comprises one or more batteries and/or an interface for receiving an Alternating Current (AC) power supply.

In one embodiment of the invention, the therapeutic device further includes a converter element, wherein the converter element is configured to operate as a step-down transformer in case of the power source delivering AC power and an inverter in case of the power source delivering Direct Current (DC) power.

In one embodiment of the invention, the PCB comprises electrical ports electrically coupled to the PCB, such that the transmitter induction coil is configured to be detachably attached to the electrical ports.

According to a second aspect of the present invention, there is provided a therapeutic device. The therapeutic device includes a first substructure including a Printed Circuit Board (PCB), a transmitter induction coil electrically coupled to the PCB, and a power source electrically coupled to the transmitter induction coil. The therapeutic device further includes one or more second substructures including one or more of respective stimulation elements and one or more respective receiver induction coils electrically coupled to the one or more respective stimulation elements. Also, the therapeutic device includes a wearable element, such that, the one or more second substructures are installed with the wearable element. The one or more stimulation elements are configured to be facing towards a body portion of a user, when in use. Furthermore, the one or more receiver induction coils are configured to receive power from a magnetic field generated by the transmitter induction coil. Also, the wearable element is embodied as a wearable pad. The wearable pad includes an outer layer including a first surface facing towards the ambient and a second surface facing opposite to the first surface. The wearable pad further includes an inner layer coupled to the outer layer. The inner layer includes a third surface facing towards the outer layer and a fourth surface configured to be facing towards the body portion of the user when in use. Also, the inner layer includes one or more slots cut into the inner layer, the one or more second substructures removably located in the one or more slots, such that, the one or more second substructures are configured to translate along the one or more slots.

In one embodiment of the invention, the transmitter induction coil defines a transmitter outer periphery. The one or more second substructures are located within the transmitter outer periphery, in one or more respective second planes parallel and/or coincident with a first plane of the transmitter outer periphery.

According to a third aspect of the present invention, there is provided a method for manufacturing a therapeutic device. The method includes steps of fabricating a first substructure by fabricating a Printed Circuit Board (PCB), fabricating a transmitter induction coil and electrically coupling the transmitter induction coil with the PCB, and electrically coupling a power source with the transmitter induction coil. The method further includes fabricating one or more second substructures including one or more of respective stimulation elements and one or more respective receiver induction coils electrically coupled to the one or more respective stimulation elements. The method further includes locating the one or more second substructures, such that, the one or more receiver induction coils are configured to receive power from a magnetic field generated by the transmitter induction coil. Also, one or more stimulation elements are configured to be facing towards a body portion of a user, when in use.

In one embodiment of the invention, the transmitter induction defines a transmitter outer periphery, and the method further includes locating the one or more second substructures within the transmitter outer periphery, in one or more respective second planes parallel and/or coincident with a first plane of the transmitter outer periphery.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of a therapeutic device (hereinafter also referred to as “the device”), in accordance with an embodiment of the present invention;

FIG. 1B illustrates an exploded view of the device in FIG. 1A

FIG. 2A illustrates an exploded view of a wearable element of the device of FIG. 1A;

FIG. 2B illustrates a bottom view of the wearable element of FIG. 2A;

FIG. 2C illustrates a partial perspective view of a section of the wearable element, in accordance with an embodiment of the present invention;

FIG. 3 illustrates an exploded view of a detachable housing of the device of FIG. 1A;

FIG. 4 illustrates a perspective view of a therapeutic device, in accordance with another embodiment of the present invention;

FIG. 5 illustrates an exploded view of the detachable housing of the device of FIG. 4;

FIG. 6 illustrates an exploded view of a therapeutic device, in accordance with yet another embodiment of the present invention;

FIG. 7 illustrates a perspective view of a therapeutic device embodied as an armband or a waist-belt, in accordance with an embodiment of the invention;

FIG. 8 illustrates a front view of a therapeutic device embodied as a face mask, in accordance with an embodiment of the present invention;

FIG. 9 illustrates a user wearing two embodiments of the therapeutic device of FIG. 7, the therapeutic device embodied as the armband and the waist-belt; and

FIG. 10 illustrates a method for manufacturing a therapeutic device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide therapeutic device (hereinafter also referred to as “the device”). The device is envisaged to be embodied in several wearable forms such as armbands, waist-belts, tops, jackets, lowers, gloves, headbands, facemasks etc. In construction, the device includes a primary substructure including a Printed Circuit Board (PCB), a transmitter induction coil electrically coupled to the PCB, and a power source (such as a battery or an AC power socket) electrically coupled to the power source. Therefore, a converter element may also be coupled to the PCB. The converter element may be a step-down transformer in case of the power source delivering AC power. Alternately, the converter element may be an inverter in case of the power source delivering Direct Current (DC) power. In several embodiments of the invention, the transmitter induction coil may be detachably attached to the PCB using electrical ports coupled to the PCB. The transmitter induction coil defines an outer periphery and one or more second substructures are envisaged to be located within the outer periphery of the transmitter induction coil. Each one of the second substructures includes a stimulation element and a receiver induction coil electrically coupled to the stimulation element. In that regard, the stimulation elements may be selected from a group consisting of irradiation sources, heating elements, cooling elements, vibration elements, electrodes and combinations thereof. While the stimulation elements are configured to facing towards a body portion of a user, the receiver induction coils are configured to receive power from a magnetic field generated by the transmitter induction coil, and transmit the received power to the corresponding stimulation elements electrically coupled to the receiver induction coils.

In several embodiments of the invention, the device may further include a wearable element and the one or more second substructures may be installed with the wearable element. In several embodiment, even the first substructure may be installed with the wearable element, however, in several alternate embodiments, the first substructure may be provided within a detachable housing configured to be detachably attached with the wearable element. For example, the wearable element may be embodied as a wearable pad. The wearable pad, in that regard, may include an outer layer, an inner layer and the one or more second substructures may be disposed between the outer layer and the inner layer. The wearable pad in turn may be embodied as one or more of a headband, a face mask, a neck belt, a body pad, a bandage, a waist belt, a shin guard, an ankle pad, a sole of a footwear, and combinations thereof.

Furthermore, the inner layer may include one or more slots cut into the inner layer, such that the second substructures are removably located in the one or more slots. Also, the one or more slots may allow the second substructures to translate along the one or more slots, thereby changing regions where the stimulation elements may be acting on the body portion of the user. The translation of the second substructures may be achieved manually, using actuators or through combinations of manual and automatic actuation. The actuators may be selected from hydraulic, pneumatic or electrical actuators. The slots may be distributed in accordance with a predetermined distribution pattern. The predetermined distribution pattern may manifest as uniform distribution of slots in a two-dimensional plane, or there may be several sectors of varying densities of slots in the predetermined distribution pattern. The sectors may overlap with many body regions of the body portions. Also, in several embodiments, the inner layer may be made up of a transparent silicone material or any other compatible diaphanous material.

In several embodiments of the invention, the first substructure may include a mode selector (for example, a resistive potentiometer or a solid state potentiometer). In that regard, the mode selector may be configured to switch between a plurality of predefined operating modes of the one or more stimulation elements. For example, the mode selector may be configured to modify a frequency and a root-mean-square (rms) value of an amplitude of electrical potential to be applied across the transmitter induction coil, thereby changing the frequency and rms value of the amplitude of the electrical potential applied across the receiver induction coils. Alternately, the first substructure may include a control module. The control module may further include a memory unit coded with machine-readable instructions and a processor configured to execute the machine-readable instructions. On execution of the machine-readable instructions, the processor may be capable of controlling supply of the electrical power to the transmitter induction coil to switch between the plurality of predefined operating modes of the one or more stimulation elements. The plurality of operating modes may correlate to changing color or wavelengths of the electromagnetic radiation emitted by the irradiation sources (for example, energizing different combinations of terminals of RGB LEDs), temperatures maintained by heating or cooling elements, frequency and amplitude of vibration provided by the vibration elements, and combinations thereof.

In that regard, for operating the one or more stimulation elements in an operating mode, the processor may be further enabled to receive a control input corresponding to the operating mode, from a user interface. The control input may switch between the plurality of predefined operating modes of the one or more stimulation elements. For example, the control input may include a frequency and a root-mean-square (rms) value of an amplitude of electrical potential to be applied across the transmitter induction coil and/or the one or more receiver induction coils. In addition, the plurality of operating modes may correlate to changing color or wavelengths of the electromagnetic radiation emitted by the irradiation sources (for example, energizing different combinations of terminals of RGB LEDs), temperatures maintained by heating or cooling elements, frequency and amplitude of vibration provided by the vibration elements, and combinations thereof. Alternatively, the processor may receive the control input from a user computing device, via a communication interface of the control module.

Embodiments of the present invention also provide a method for manufacturing a therapeutic device. The method includes fabricating the first substructure by first fabricating the PCB and the transmitter induction coil. The transmitter induction coil may then be electrically coupled to the PCB (detachably or permanently) through electrical ports provided with the PCB. Furthermore, the power source may be electrically coupled to the PCB and the transmitter induction coil. Furthermore, the one or more second substructures may then be fabricated. Each one of the secondary substructures may be provided with a receiver induction coil and a stimulation element electrically coupled to the receiver induction coil. Also, the one or more second substructures may be located such that the one or more receiver induction coils are configured to receive power from a magnetic field generated by the transmitter induction coil. In several embodiments of the invention, the one or more second substructures may be located within the transmitter outer periphery defined by the transmitter induction coil, such that, the one or more respective stimulation elements are configured to be facing towards the body portion of the user.

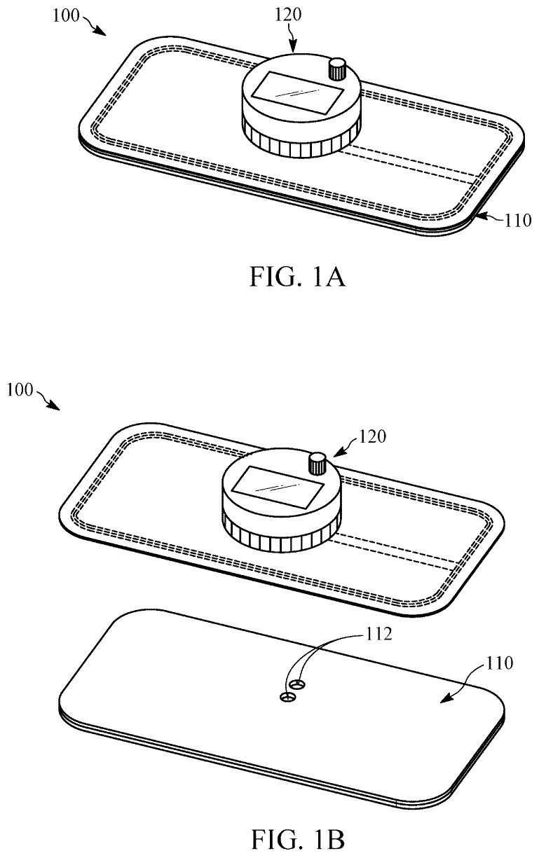

Several embodiments of the present invention will now be discussed in detail taking FIGS. 1A-10 as reference.

FIG. 1A illustrates a perspective view of a therapeutic device 100 (hereinafter also referred to as “the device 100”), in accordance with an embodiment of the present invention. The device 100 includes a wearable element 110 and a detachable housing 120. The wearable element 110 is envisaged to be made up of flexible materials so that the wearable element 110 can wrap around several body portions of a user. The wearable element 110 has been illustrated in a form of a wearable pad and may be embodied as one or more of a headband, a face mask, a neckband, a body pad, a bandage, a waist belt, a shin guard, an ankle pad, a sole of footwear, and combinations thereof. The detachable housing 120 includes the power and control circuitry of the device 100.

FIG. 1B illustrates an exploded view of the device in FIG. 1A. As illustrated in FIG. 1B, the detachable housing 120 is detachably attached to the wearable element 110. In that regard, at least two connection ports 112 have been provided with the wearable element 110. The at least two connection ports 112 act as electrical terminals for connecting the power and the control circuitry to the wearable element 110. In that regard, the wearable element 110 may be provided with conductive contact discs at a predetermined depth at the at least two connection ports 112 and the detachable housing 120 may include protruding spring loaded dowel pins configured to make physical contact with the conductive contact discs. In several embodiments of the invention, the wearable element 110 may be provided with four (4) connection ports 112 thereby preventing relative motion between the detachable housing 120 and the wearable element 110 when the detachable housing 120 and the wearable element 110 are attached to each other.

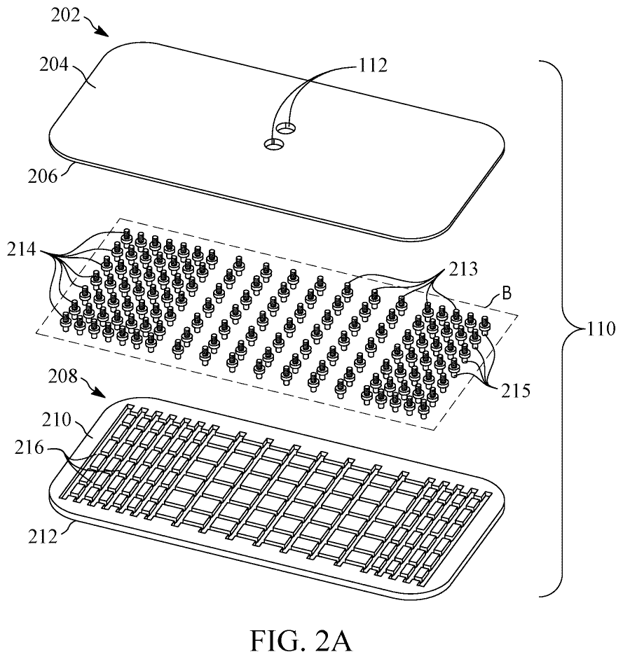

FIG. 2A illustrates an exploded view of the wearable element 110 of the device 100 of FIG. 1A. The wearable element 110 includes an outer layer 202. The outer layer 202 may be made up of a flexible opaque material to prevent leakage of radiation generated by irradiation sources of the device 100, towards the ambient. The outer layer 202 includes a first surface 204 facing towards the ambient. The outer layer 202 further includes a second surface 206 facing opposite to the first surface 204. The wearable element 110 further includes an inner layer 208. The inner layer 208 includes a third surface 210 facing towards the outer layer 202, and a fourth surface 212 configured to face the body portion of the user, when the wearable element 110 is in use. The inner layer 208 is configured to be coupled to the outer layer 202.

The inner layer 208 includes one or more slots 216 cut into the inner layer 208. The one or more slots 216 have one or more respective predetermined depths from either the third surface 210 or the fourth surface 212. In several embodiments of the invention, the one or more slots 216 may be evenly distributed over the fourth surface 212. In several alternate embodiments of the invention, the one or more slots 216 may be distributed in accordance with a predetermined distribution pattern (See FIG. 2B).

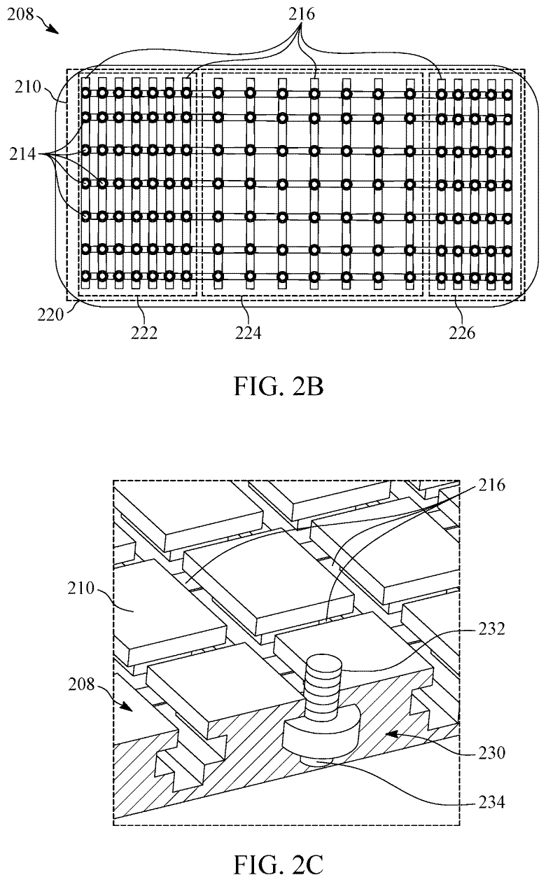

The wearable element 110 further includes one or more second substructures 214 located between the outer layer 202 and the inner layer 208. Further, the one or more second substructures 214 are located in a second plane B sectioning each one of the one or more second substructures 214. Although, all of the one or more second substructures 214 are illustrated to be in a single second plane B, in several alternate embodiments, the one or more substructures 214 may be located in one or more distinct or coincident second planes, all of the second planes being parallel or coincident with a first plane of a transmitter induction coil (See FIG. 3). The one or more second substructures 214 includes one or more respective stimulation elements 215 electrically coupled to one or more respective receiver induction coils 213. The one or more second substructures 214 are configured to have the one or more respective stimulation elements 215 facing towards the body portion of the user when the device 100 is in use (See FIG. 2C). The one or more respective stimulation 215 elements in that regard may be selected from a group consisting of irradiation sources, heating elements, cooling elements, vibration elements, electrodes and combinations thereof. Furthermore, irradiation sources may be selected from a group consisting of Light Emitting Diodes (LEDs), and lasers. Additionally, the irradiation sources may be configured to operate in one or more of a pulse mode and a continuous mode. In case of a stimulation element being an irradiation source, the stimulation element would be emitting the electromagnetic radiation. In the case of a stimulation element being a vibration element, the stimulation element would be a vibrating head connected to an eccentric mass rotating motor or a linear resonant motor.

In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the body of a user.

TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

FIG. 2B illustrates a bottom view of the wearable element 110 of FIG. 2A. As illustrated in FIG. 2B, the one or more slots 216 is divided in accordance with a predetermined distribution pattern 220. Furthermore, the predetermined distribution pattern 220 includes one or more sectors 222, 224, and 226. In several embodiments of the invention, the one or more sectors 222, 224, and 226 are configured to overlap with one or more body regions of the body portion of the user on which the wearable element 110 has been worn. In that regard, the density or number of slots for a unit value of area may be different in different sectors of the one or more sectors 222, 224, and 226. For example, if the device 100 is embodied as a face mask, there may be provided on the wearable element 110 sectors configured to overlap regions such as just under the eyes, forehead, checks, and chin of the user where the density of slots may be relatively higher when compared with other sectors of the predetermined distribution pattern 220 of the device 100 embodied as the face mask.

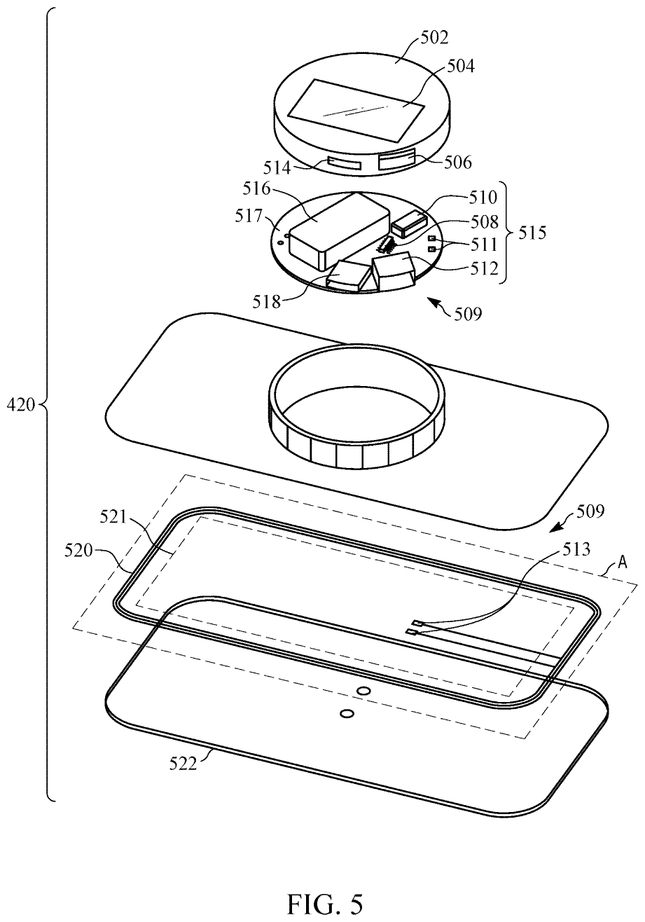

FIG. 2B also illustrates the one or more second substructures 214 located in the one or more slots 216, such that, the one or more second substructure 214 are configured to translate along the one or more slots 216. In several embodiments of the invention, the one or more second substructures 214 may be removably located in the one or more slots 216. The removable location of the one or more second substructures 214 allows for only necessary number or count of second substructures 214 to be installed in the wearable element 110, thereby saving power when portions of the wearable element 110, not covering a targeted body region of the user, need not be activated. In several embodiments of the invention, the one or more second substructures 214 may be configured to be translated along the one or more slots 216 through manual manipulation, electrical actuation (See FIG. 6), and combinations thereof. The translation of the one or more substructures 214 along the one or more slots 216 allows for concentration of respective one or more stimulation elements 215 in a particular target body portion or body region of the user for a more concentrated and combinational therapeutic effect without having to add any further second substructures.

FIG. 2C illustrates a partial perspective view of a section of the wearable element 110, in accordance with an embodiment of the present invention. FIG. 2C illustrates one or more slots 216 cut into the inner layer 208 and provided at a predetermined depth from the third surface 210. Also, a second substructure 230 of the one or more second substructures 214 is located in one of the one or more slots 216. The second substructure 230 includes a receiver induction coil 232 of the one or more receiver induction coils 213 corresponding to the one or more respective second substructures 214. Also, the second substructure 230 includes a stimulation element 234 as discussed in the preceding discussion.

FIG. 3 illustrates an exploded view of the detachable housing 120 of the device 100 of FIG. 1A. The detachable housing 120 includes an upper portion 306 and a lower portion 320. The upper portion 306 may be configured to be attached to the lower portion 320 using fasteners such as screws and snap-fit arrangements. The upper portion 306 includes a user interface 302 that may include a capacitive or resistive touch-based interface and/or several tactile buttons for activation and deactivation of the device 100 or for changing modes of operation of the device 100, etc. The user interface 302 may also include an LCD or LED based display for indicating the status of device 100, an active mode of operation of the device 100, remaining electrical power in a power source 312 of the device 100 located within the detachable housing 120, and combinations thereof. The power source 312 may further include one or more replaceable batteries or an interface to connect with the AC power supply.

The upper portion 306 also includes a control knob 304 that is mechanically coupled to a mode selector 308 configured for to switching between a plurality of predefined operating modes of the one or more stimulation elements 215. For example, the mode selector 308 may be configured for modifying a frequency and a root-mean-square (rms) value of an amplitude of electrical potential to be applied across terminals 311 of a transmitter induction coil 316 and consequently modifying the frequency and the root-mean-square (rms) value of the amplitude of the electrical potential applied across the transmitter induction coil 316. The mode selector 308 in that regard may be a resistive or a solid-state potentiometer. In addition, the plurality of operating modes may correlate to changing color or wavelengths of the electromagnetic radiation emitted by the irradiation sources (for example, energizing different combinations of terminals of RGB LEDs), temperatures maintained by heating or cooling elements, frequency and amplitude of vibration provided by the vibration elements, and combinations thereof. In that regard, the device 100 may be programmed to predefine the plurality of operating modes corresponding to a plurality of positions of a marker provided on the mode selector 308 or the control knob 304.

The transmitter induction coil 316 has been provided in a first plane A and defines a transmitter outer periphery 317. The first plane A may be parallel to the one or more second planes and/or the second plane B. The one or more receiver induction coils 214 are configured to receive power from a time-varying magnetic field generated by the transmitter induction coil 316. Therefore, it is envisaged that the one or more second substructures 214 are located within the transmitter outer periphery 317. The power source 312, the PCB 314, and the transmitter induction coil 316 together constitute a first substructure 313 essential for functioning of the device 100. The user interface 302, the power source 312, and the mode selector 308 may be electrically coupled to a Printed Circuit Board (PCB) 314. The PCB 314 further includes a converter element 310. In the case of the power source 312 being the interface for connecting the AC power supply, the converter element 310 may be a step-down transformer. Alternately, in the case of the power source 312 including batteries capable of delivering Direct Current (DC) power, the converter element 310 may be an inverter for converting DC into AC to enable the transmitter induction coil 316 to generate the time-varying magnetic field. In several embodiments of the invention, the PCB 314 also includes electrical ports 309 for detachably attaching terminals 311 of the transmitter induction coil 316.

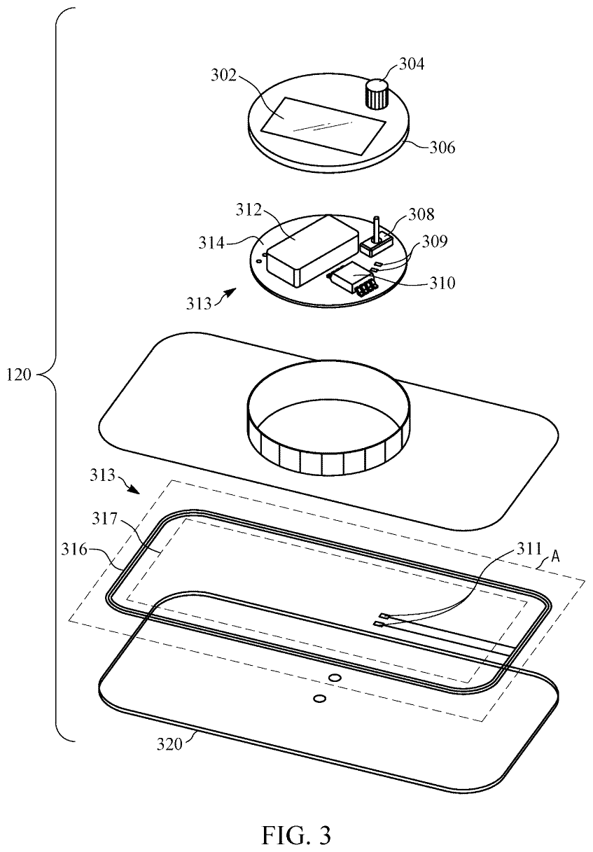

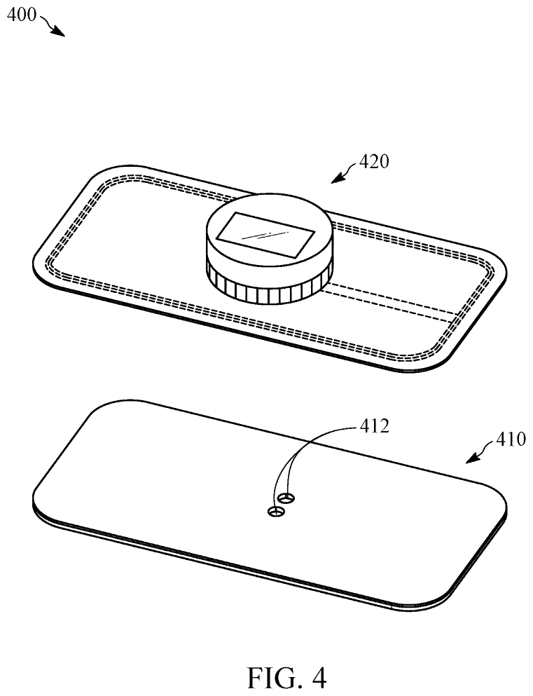

FIG. 4 illustrates a perspective view of a therapeutic device 400 (hereinafter also referred to as “the device 400”), in accordance with another embodiment of the present invention. The device 400 includes a wearable pad 410 which may have an identical construction to any one of the several embodiments of the wearable element 110. Further, the wearable pad 410 includes at least two connection ports 412. Furthermore, the device 400 includes a detachable housing 420. FIG. 5 illustrates an exploded view of the detachable housing 420 of the device of FIG. 4. The detachable housing 420 includes an upper portion 502 and a lower portion 522 configured to be attached using one or more fasteners such as screws, snap-fit arrangements, internal-external thread combinations, etc. Furthermore, the upper portion 502 includes a user interface 504, a first cutout 506 for receiving a data transfer cable, and a second cutout 514 for receiving a power cable.

The detachable housing 420 further includes a power source 516 that may be an AC or DC power source as discussed in the context of the power source 312. The user interface 504 and the power source 516 are electrically coupled to a PCB 517. Further electrically coupled to the PCB 517 is a power port 518. The power port 518 may be an AC power connection port where a power cable from an AC plug or a charging brick may be connected. Further electrically coupled to the PCB 517 are a processor 508, a memory unit 510, electrical ports 511, and a communication interface 512. The processor 508, the memory unit 510 and the communication interface 512 together constitute a control module 515. The electrical ports 511 allow for detachably attaching terminals 513 of a transmitter induction coil 520 defining a transmitter outer periphery 521 in the first plane A. The power source 516, the PCB 517, and the transmitter induction coil 520 together constitute a first substructure 509. The memory unit 510 includes machine-readable instructions that when executed by the processor 508, enable the processor 508 to switch between the plurality of predefined operating modes of the one or more stimulation elements 215.

In several embodiments of the invention, for operating the one or more stimulation elements 215 in an operating mode, the processor 508 is further enabled to receive a control input corresponding to the operating mode, from the user interface 504. For example, the control input may include a frequency and a root-mean-square (rms) value of an amplitude of electrical potential to be applied across the transmitter induction coil 520 and/or the one or more receiver induction coils 213. In addition, the plurality of predefined operating modes may correlate to changing color or wavelengths of the electromagnetic radiation emitted by the irradiation sources (for example, energizing different combinations of terminals of RGB LEDs), temperatures maintained by heating or cooling elements, frequency and amplitude of vibration provided by the vibration elements, and combinations thereof. In several embodiments of the invention, the plurality of predefined modes of the one or more stimulation elements 215 may be mapped with a plurality of respective control values, in the memory unit 510. The control signal therefore may include a specific control value corresponding to a desired predefined operating mode of the one or more stimulation elements 215. In several alternate embodiments, for operating the one or more stimulation elements in the predefined operating mode of the plurality of predefined operating modes, the processor 508 is further enabled to receive the control input corresponding to the predefined operating mode, from a user computing device (not shown), via the communication interface 512 of the control module 515.

FIG. 6 illustrates an exploded view of a therapeutic device 600 (hereinafter referred to as “the device 600”), in accordance with yet another embodiment of the present invention. The device 600 illustrated in FIG. 6 is a unitary device, i.e., all the components of the device 600 are contained within a single construct without having a detachable element that can be detached and stored separately or used in the context of another identical device without having to disassemble the device 600 as would be required during repair and/or reconstruction. The device 600 includes an outer layer 602 and an inner layer 630. On a surface disposed to the ambient, the outer layer 602 includes a user interface 608 and a dial 604. Further, on a lateral surface, the outer layer 602 includes a first cutout 606 configured to receive a data transfer cable and a second cutout 618 configured to receive a power cable.

Furthermore, located between the outer layer 602 and the inner layer 630 is a power source 620 electrically coupled to a PCB 621. The user interface 608 is also electrically coupled to the PCB 621. Further, the PCB 621 is envisaged to be a flexible PCB. Further electrically coupled to the flexible PCB 621 is a power port 622, one or more processors 609, a memory unit 610, a mode selector 614 mechanically coupled to the dial 604, electrical ports 612 configured to detachably attach terminals 634 of a transmitter induction coil 624, and a communication interface 616. The power source 620, the PCB 621, and the transmitter induction coil 624 together constitute a first substructure 623. The transmitter induction coil 624 defines a transmitter outer periphery 625 in a first plane A. The one or more processors 609, the memory unit 610, and the communication interface 616 together constitute a control module 615. Further located between the outer layer 602 and the inner layer 630 is an electrical actuator 626. The electrical actuator 626 may be a rotary motor or a linear electromagnetic motor. The electrical actuator 626 as discussed above is configured to achieve electrically actuated translation of one or more second substructure 628 along one or more slots 632 cut into the inner layer 630. The one or more second substructures 628 are located within the transmitter outer periphery 625 in the second plane B. The second plane B may be parallel or coincident with the first plane A.

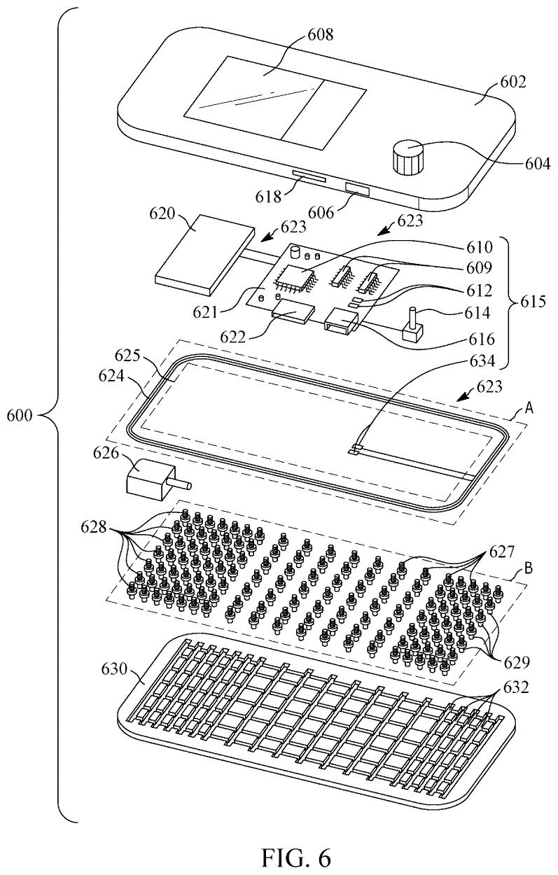

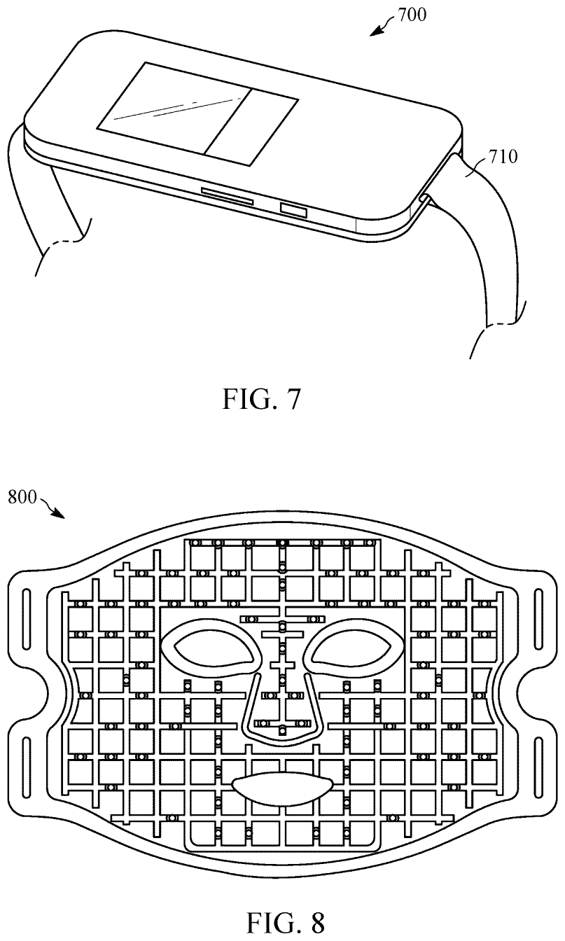

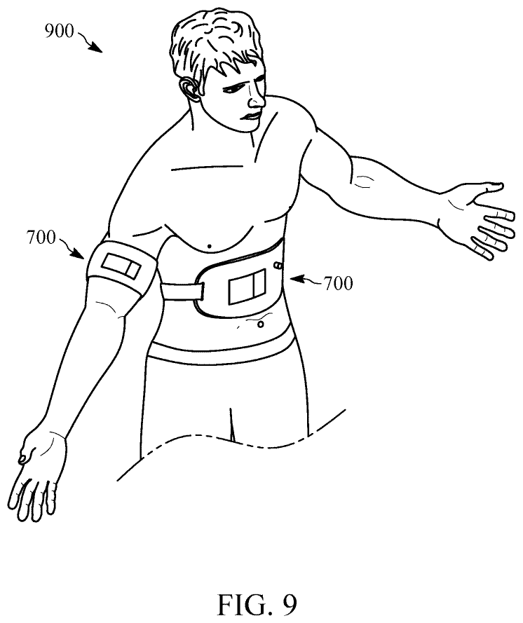

FIG. 7 illustrates a perspective view of a therapeutic device 700 embodied as an armband or a waist-belt, in accordance with an embodiment of the invention. In that regard, the therapeutic device 700 includes fastening means 710 that may include straps, belts, or combinations of straps, belts, buckles, and clips. FIG. 8 illustrates a front view of a therapeutic device 800 embodied as a face mask, in accordance with an embodiment of the present invention. FIG. 9 illustrates a user 900 wearing two embodiments of the therapeutic device 700 of FIG. 7. The therapeutic device 700 is embodied as the armband and the waist belt.

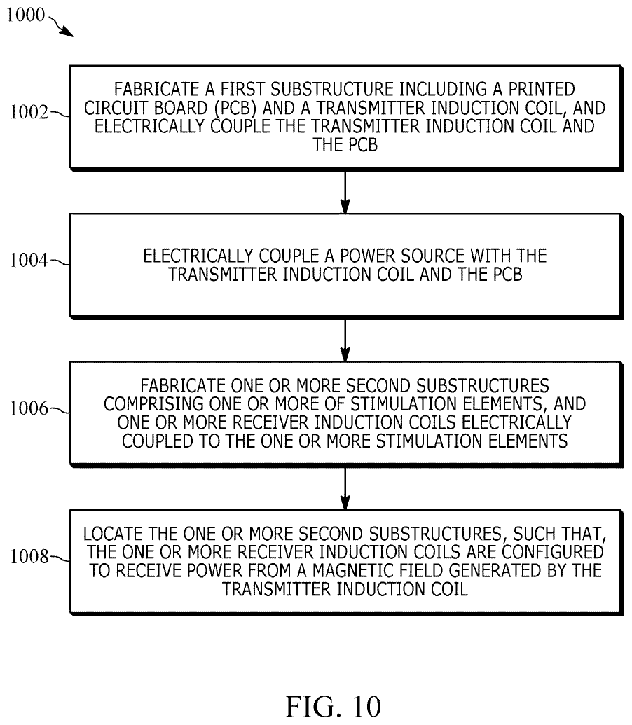

FIG. 10 illustrates a method 1000 for manufacturing a therapeutic device 100, 400 or 600, in accordance with an embodiment of the present invention. The method 1000 begins at Step 1002 by fabricating the first substructure 313, 509, or 623. For fabricating the first substructure 313, 509, or 623, the PCB 314, 517 or 621 is fabricated. Further, the transmitter induction coil 316, 520, or 624 is fabricated. In several embodiments of the invention, the transmitter induction coil 316, 520, or 624 defines the transmitter outer periphery 317, 521, or 625, respectively. At Step 1004, the transmitter induction coil 316, 520, or 624 and the power source 312, 516, or 620 are then electrically coupled to the PCB 314, 517 or 621. At Step 1006, the one or more second substructures 214 or 628 are fabricated. The one or more second substructures 214 or 628 include the one or more respective receiver induction coils 213 or 627 electrically coupled to the one or more respective stimulation elements 215 or 629. At Step 1008, the one or more second substructures 214 or 628 are located such that the one or more receiver induction coils 213 or 627 are configured to receive power from a magnetic field generated by the transmitter induction coil 316, 520, or 624. In several embodiments of the invention, the one or more second substructures 214 or 628 are located within the transmitter outer periphery 317, 521, or 625, in the one or more respective second planes A parallel and/or coincident with the first plane B of the transmitter outer periphery 317, 521, or 625. Also, the one or more stimulation elements 215 or 629 are configured to be facing towards a body portion of a user, when in use.

The embodiments of the therapeutic device offer several advantages. For instance, the device requires minimal wiring as the stimulation elements are powered wirelessly using coupling of respective receiver induction coils with a time-varying magnetic field generated by a transmitter induction coil, thereby making the device less material-intensive and lighter in weight. Furthermore, a single device can be used to provide combination therapy involving several kinds of therapeutic and recreational effects such as irradiation, heating, cooling, vibratory massage, and electrical stimulation. The slots may be distributed in such a manner as to allow specific muscle groups, blood vessels, and organ systems to be targeted for therapy. Also, intensity and other operational parameters of the therapy may be modified using dials, user interfaces or wirelessly using user computing devices such as smartphones, tablet PCs, desktop PCs, notebook PCs, and the like.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic device, comprising:
a first substructure comprising a Printed Circuit Board (PCB), a transmitter induction coil electrically coupled to the PCB, and a power source electrically coupled to the transmitter induction coil; and
one or more second substructures comprising one or more of respective stimulation elements and one or more respective receiver induction coils electrically coupled to the one or more respective stimulation elements,
wherein the one or more stimulation elements are configured to be facing towards a body portion of a user, when in use, and
wherein the one or more receiver induction coils are configured to receive power from a magnetic field generated by the transmitter induction coil.

2. The therapeutic device as claimed in claim 1, wherein the transmitter induction coil defines a transmitter outer periphery and the one or more second substructures are located within the transmitter outer periphery, in one or more respective second planes parallel and/or coincident with a first plane of the transmitter outer periphery.

3. The therapeutic device as claimed in claim 1, wherein the one or more stimulation elements are selected from a group consisting of irradiation sources, heating elements, cooling elements, vibration elements, electrodes and combinations thereof.

4. The therapeutic device as claimed in claim 3, wherein the irradiation sources are selected from a group consisting of Light Emitting Diodes (LEDs), and lasers.

5. The therapeutic device as claimed in claim 1, further comprising a wearable element, such that, the one or more second substructures are installed with the wearable element.

6. The therapeutic device as claimed in claim 5, wherein the first substructure is provided within a detachable housing configured to be detachably attached with the wearable element comprising the one or more second substructures.

7. The therapeutic device as claimed in claim 5, wherein the wearable element is embodied as a face mask.

8. The therapeutic device as claimed in claim 5, wherein the wearable element is embodied as a wearable pad comprising:
an outer layer comprising a first surface facing towards the ambient and a second surface facing opposite to the first surface; and
an inner layer coupled to the outer layer, the inner layer comprising a third surface facing towards the outer layer and a fourth surface configured to be facing towards the body portion of the user when in use,
wherein the one or more second substructures are located between the outer layer and the inner layer.

9. The therapeutic device as claimed in claim 8, wherein the wearable pad is embodied as one or more of a headband, a neck belt, a body pad, a bandage, a face mask, a waist belt, a shin guard, an ankle pad, a sole of a footwear, and combinations thereof.

10. The therapeutic device as claimed in claim 8, wherein the inner layer is made up of a transparent silicone material.

11. The therapeutic device as claimed in claim 8, wherein the inner layer comprises one or more slots cut into the inner layer, the one or more second substructures removably located in the one or more slots, such that, the one or more second substructures are configured to translate along the one or more slots.

12. The therapeutic device as claimed in claim 11, wherein the one or more second substructures are configured to be translated along the one or more slots through manual manipulation, electrical actuation, and combinations thereof.

13. The therapeutic device as claimed in claim 11, wherein the one or more slots are distributed in accordance with predetermined distribution pattern comprising one or more sectors configured to overlap one or more respective body regions of the body portion.

14. The therapeutic device as claimed in claim 1, wherein the first substructure further comprises a mode selector electrically coupled to the PCB, the mode selector configured to switch between a plurality of predefined operating modes of the one or more stimulation elements.

15. The therapeutic device as claimed in claim 1, wherein the first substructure further comprises a control module, the control module comprising a processor and a memory unit operably connected to the processor, the memory unit comprising machine-readable instructions that when executed by the processor, enable the processor to:
switch between a plurality of predefined operating modes of the one or more stimulation elements.

16. The therapeutic device as claimed in claim 15, wherein for operating the one or more stimulation elements in an operating mode, the processor is further enabled to receive a control input corresponding to the operating mode, from a user interface, the control input configured to switch between a plurality of predefined operating modes of the one or more stimulation elements.

17. The therapeutic device as claimed in claim 15, wherein for operating the one or more stimulation elements in an operating mode, the processor is further enabled to receive a control input corresponding to the operating mode, from a user computing device, via a communication interface of the control module, the control input configured to switch between a plurality of predefined operating modes of the one or more stimulation elements.

18. The therapeutic device as claimed in claim 1, wherein the power source comprises one or more batteries and/or an interface for receiving an Alternating Current (AC) power supply.

19. The therapeutic device as claimed in claim 18, further comprising a converter element, wherein the converter element is configured to operate as a step-down transformer in case of the power source delivering AC power and an inverter in case of the power source delivering Direct Current (DC) power.

20. The therapeutic device as claimed in claim 1, wherein the PCB comprises electrical ports electrically coupled to the PCB, such that, the transmitter induction coil is configured to be detachably attached to the electrical ports.

21. A therapeutic device, comprising:
a first substructure comprising a Printed Circuit Board (PCB), a transmitter induction coil electrically coupled to the PCB, and a power source electrically coupled to the transmitter induction coil, the transmitter induction coil;
one or more second substructures comprising one or more of respective stimulation elements and one or more respective receiver induction coils electrically coupled to the one or more respective stimulation elements; and
a wearable element, such that, the one or more second substructures are installed with the wearable element,
wherein the one or more stimulation elements are configured to be facing towards a body portion of a user, when in use,
wherein the one or more receiver induction coils are configured to receive power from a magnetic field generated by the transmitter induction coil, and
wherein the wearable element is embodied as a wearable pad comprising:
an outer layer comprising a first surface facing towards the ambient and a second surface facing opposite to the first surface, and
an inner layer coupled to the outer layer, the inner layer comprising a third surface facing towards the outer layer and a fourth surface configured to be facing towards the body portion of the user when in use,
wherein the one or more second substructures are located between the outer layer and the inner layer, and
wherein the inner layer comprises one or more slots cut into the inner layer, the one or more second substructures are removably located in the one or more slots, such that, the one or more second substructures are configured to translate along the one or more slots.

22. The therapeutic device as claimed in claim 21, wherein the transmitter induction coil defines a transmitter outer periphery and the one or more second substructures are located within the transmitter outer periphery, in one or more respective second planes parallel and/or coincident with a first plane of the transmitter outer periphery.

23. A method for manufacturing a therapeutic device, the method comprising steps of:
fabricating a first substructure by:
fabricating a Printed Circuit Board (PCB),
fabricating a transmitter induction coil,
electrically coupling the transmitter induction coil with the PCB, and
coupling, electrically, a power source with the transmitter induction coil and the PCB;
fabricating one or more second substructures comprising one or more of respective stimulation elements and one or more respective receiver induction coils electrically coupled to the one or more respective stimulation elements; and
locating the one or more second substructures, such that, the one or more receiver induction coils are configured to receive power from a magnetic field generated by the transmitter induction coil,
wherein the one or more stimulation elements are configured to be facing towards a body portion of a user, when in use.

24. The method as claimed in claim 23, wherein the transmitter induction defines a transmitter outer periphery, and the method further includes locating the one or more second substructures within the transmitter outer periphery, in one or more respective second planes parallel and/or coincident with a first plane of the transmitter outer periphery.
